(19) United States
(12) Patent Application Publication
(10) Pub. No.: US 2020/0407679 A1
(43) Pub. Date: Dec. 31, 2020

(54) CELL GROWTH METHOD, CELL GROWTH AGENT AND CELL GROWTH MEDIUM

(71) Applicant: PANAGY CO., LTD., Tokyo (JP)

(72) Inventors: Shoji KOGA, Tokyo (JP); Toshimitsu ITAYA, Tokyo (JP)

(73) Assignee: PANAGY CO., LTD., Tokyo (JP)

(21) Appl. No.: 17/018,030

(22) Filed: Sep. 11, 2020

Related U.S. Application Data
(63) Continuation of application No. PCT/JP2020/017493, filed on Apr. 23, 2020.

(30) Foreign Application Priority Data
May 29, 2019 (JP) 2019-100248
Dec. 5, 2019 (JP) 2019-220646

(51) Int. Cl. C12N 5/00; C12N 5/077; C12N 5/071

(52) U.S. Cl. CPC C12N 5/0037; C12N 5/0656; C12N 2502/1364; C12N 2500/90; C12N 5/0629

(57) ABSTRACT

Provided are a cell growth method including serum-free culture of somatic cells sowed in a cell growth medium containing a culture supernatant of dental pulp stem cells, wherein the somatic cells exclude physically or physiologically defected somatic cells, is a novel cell growth method for serum-free culture of somatic cells.

### CROSS-REFERENCE TO RELATED APPLICATIONS

This application is a Continuation of PCT International Application No. PCT/JP2020/017493, filed on Apr. 23, 2020, which claims priority under 35 U.S.C. Section 119(a) to Japanese Patent Application No. 2019-100248 filed on May 29, 2019 and Japanese Patent Application No. 2019-220646 filed on Dec. 5, 2019. Each of the above applications is hereby expressly incorporated by reference, in its entirety, into the present application.

### BACKGROUND OF THE INVENTION

### Field of the Invention

The present invention relates to a cell growth method, a cell growth agent and a cell growth medium.

### Description of the Related Art

As a cell growth method, a method for cell growth using a stem cell culture liquid is known (for example, see JP 2015-186474 A and JP 2018-23343 A).

JP 2015-186474 A describes a cell culture method including a sowing step of singly sowing single cells on a coating layer of microchannels having a laminin-containing coating layer on the inner wall thereof, and a circulation step of circulating a culture liquid inside the microchannels sowed singly with the single cells in the sowing step.

JP 2018-23343 A describes a medium for cell growth culture that contains MIG and I-309.

### CITATION LIST

PTL 1: JP 2015-186474 A
PTL 2: JP 2018-23343 A
PTL 3: WO2005/026343
PTL 4: CN 105861429
PTL 5: CN 107475188
PTL 6: CN 105420186
PTL 7: CN 105238749

### SUMMARY OF THE INVENTION

Recently, in the regenerative medicine area, it is desired to use cells obtained by growing culture for disease treatment. In the case of using cells obtained by growing culture for disease treatment, from the viewpoint of morality and safety, preferably, the cells obtained by growing culture do not contain serum. Accordingly, as a cell growth agent for growing culture of somatic cells, a serum substitute for serum-free culture of somatic cells is desired more.

However, JP 2015-186474 A and JP 2018-23343 A do not clearly describe use of a culture supernatant of mesenchymal stem cells as a serum substitute for serum-free culture of somatic cells.

On the other hand, as a neurosphere method, there is known a method of growing neural stem cells while being differentiated by adding a culture supernatant of neural stem cells to a culture liquid. WO2005/026343 describes a method of promoting life, growth or both the two of neural stem cells in a culture liquid, which includes a step of overexpressing galectin-1 in neural stem cells. Such a neurosphere method is not expected to be applicable to a culture supernatant of mesenchymal stem cells (dental pulp stem cells) that are not neural stem cells, or applicable to use as a serum substitute for serum-free culture of arbitral somatic cells.

Also there is known a method of growing various stem cells using a culture supernatant of various stem cells (see CN 105861429, CN 107475188, CN 105420186 and CN 105238749).

CN 105861429 describes that, when dental mesenchymal stem cells after cryopreservation are incubated in a medium prepared by adding a low-temperature preservation liquid in an amount of five times to a culture supernatant obtained by incubation of dental mesenchymal stem cells in a serum-free DMEM/F12 medium, the cell growth after thawing betters (Examples, FIG. 1).

CN 107475188 describes incubation of adipose stem cells by adding a bFGF-containing serum-free medium thereto, followed by collection of the culture supernatant after 24-hour incubation, demonstrating that, when embryonic stem cells are incubated in a cell medium containing 75% of the supernatant, the cell growth is promoted as compared with the control incubated in a medium not containing the supernatant (Summary, Examples).

CN 105420186 describes a serum-free stem cell incubation kit containing 4 to 6 parts by weight of a concentrated culture supernatant of umbilical mesenchymal stem cells (Summary, Claims). Specifically, this demonstrates that, when human umbilical mesenchymal stem cells are incubated in a serum-free medium (SCL-M) and a serum medium (FBS) containing 5 parts by weight of a concentrated culture supernatant of umbilical mesenchymal stem cells prepared by sowing human umbilical mesenchymal stem cells in a serum-free medium and incubating them therein for 48 to 72 hours, and then collecting and centrifuging the culture supernatant, the cell growth is promoted more in culture of the cells in SCL-M (Example 5, FIG. 3).

CN 105238749 describes that, when bone marrow mesenchymal stem cells after cryopreservation are incubated in a medium prepared by adding a low-temperature preservation liquid in an amount of five times to a culture supernatant obtained by incubation of bone marrow mesenchymal stem cells in a serum-free DMEM medium, the cell growth after thawing betters (Examples, FIG. 1, FIG. 2).

However, in CN 105861429, CN 107475188, CN 105420186 and CN 105238749, use of a culture supernatant of various types of stem cells as a serum substitute for serum-free culture of somatic cells is not anticipated. Example 1 of CN 105861429 describes “30-age or younger human healthy deciduous teeth and evulsion teeth are collected”, merely stating use of a culture supernatant of permanent tooth pulp mesenchymal stem cells.

A technical problem of the present invention is to provide a novel cell growth method and cell growth agent for serum-free culture of somatic cells.

As a result of assiduous studies made for the purpose of solving the above-mentioned problems, the present inventors have found that serum-free culture of somatic cells sowed in a cell growth medium containing a culture supernatant of dental pulp stem cells can solve the above-mentioned problems.

Specifically, the present invention and preferred embodiments of the present invention are as follows.

[1] A cell growth method including serum-free culture of somatic cells sowed in a cell growth medium containing a culture supernatant of dental pulp stem cells.
[1-1] The cell growth method according to [1], wherein the somatic cells exclude physically or physiologically defected somatic cells.
[2] The cell growth method according to [1] or [1-1], wherein only a cell growth agent containing a culture supernatant of deciduous dental pulp stem cells is used as the culture supernatant of dental pulp stem cells.
[3] The cell growth method according to any one of [1], [1-1] or [2], wherein the somatic cells are fibroblasts or epidermal keratinocytes.
[4] A cell growth agent containing a culture supernatant of deciduous dental pulp stem cells.
[4-1] A cell growth agent containing a culture supernatant of deciduous dental pulp stem cells, for use for serum-free culture of somatic cells.
[4-2] The cell growth agent according to [4-1], wherein the somatic cells exclude physically or physiologically defected somatic cells.
[5] A cell growth medium containing the cell growth agent of any one of [4], [4-1] or [4-2].
[5-1] The cell growth medium according to [5], for use for serum-free culture of somatic cells.
[5-2] The cell growth medium according to [5-1], wherein the somatic cells exclude physically or physiologically defected somatic cells.
[6] The cell growth medium according to any one of [5], [5-1] or [5-2], containing the cell growth agent in an amount of 5% by mass or more relative to the total of the cell growth medium.

According to the present invention, there can be provided a novel cell growth method and a novel cell growth agent for serum-free culture of somatic cells.

### BRIEF DESCRIPTION OF THE DRAWINGS

FIG. 1 is a graph (average of 12 samples) showing the results of quantitative assay of cell growth in Example 1 (SGF) and Comparative Example 1 (serum-free).

FIG. 2 is a graph (average of 12 samples) showing the results of quantitative assay of cell growth in Example 1 (SGF) and Comparative Example 2 (AT).

FIG. 3 is a graph (average of 9 samples) showing the results of quantitative assay of cell growth in Example 1 (SGF) and Comparative Example 3 (UC).

FIG. 4 is a graph (average of 6 samples) showing the results of quantitative assay of cell growth in Example 1 (SGF) and Comparative Example 4 (HFDM).

FIG. 5 is a graph (average of 6 samples) showing the results of quantitative assay of cell growth in Example 1 (SGF) and Comparative Example 5 (KBM).

FIG. 6 is a graph (average of 12 samples) showing the results of quantitative assay of cell growth in Example 1 (SGF) and Comparative Example 6 (FBS).

FIG. 7 is a graph showing the results of quantitative assay of cell growth in Example 101 (SGF) and Comparative Example 101 (serum-free).

FIG. 8 is a graph showing the results of quantitative assay of cell growth in Example 101 (SGF) and Comparative Example 102 (UC).

FIG. 9 is a graph showing the results of quantitative assay of cell growth in Example 101 (SGF) and Comparative Example 103 (serum-free medium).

FIG. 10 is a graph showing the results of quantitative assay of cell growth in Example 101 (SGF) and Comparative Example 104 (FBS).

FIG. 11 is a graph showing the results of quantitative assay of cell growth in Example 201 (SGF) and Comparative Example 201 (adult dental pulp).

### DESCRIPTION OF THE PREFERRED EMBODIMENTS

Hereinunder the present invention is described in detail. The description of the constituent elements given below may be based on typical embodiments and specific examples of the present invention, but the present invention is not limited to such embodiments. In this description, the numerical range expressed as (A to B) is meant to include the numerical values of A and B as a lower limit and an upper limit.

[Cell Growth Method and Cell Growth Agent]

The cell growth method of the present invention is a cell growth method that includes serum-free culture of somatic cells sowed in a cell growth medium containing a culture supernatant of dental pulp stem cells.

On the other hand, the cell growth agent of the present invention is a cell growth agent that contains a culture supernatant of deciduous dental pulp stem cells. The cell growth agent of the present invention is preferably for use for serum-free culture of somatic cells. Hereinunder, preferred embodiments of the cell growth method and the cell growth agent of the present invention are described.

In embodiments of the cell growth method, the cell growth agent and the cell growth medium of the present invention, the somatic cells may exclude physically or physiologically defected somatic cells. WO2011/118795 described in [0018] as follows: “A damaged area’ means an area of a tissue that could not exhibit the original function thereof owing to physical or physiological defects having occurred in the tissue, and is used as a concept to include not only external injuries but also lesioned parts, damaged parts or diseased parts caused by physical or physiological defects of tissues”. One preferred embodiment of the present invention is a cell growth method for serum-free culture of somatic cells, and this embodiment quite differs from the use invention of “repairing a damaged part of a target tissue (cells)” described in WO2011/118795.

<Cell Growth Medium>

The cell growth method of the present invention uses a cell growth medium containing a culture supernatant of dental pulp stem cells.

(Culture Supernatant of Dental Pulp Stem Cells)

The cell growth medium contains a culture supernatant of dental pulp stem cells.

Preferably, the culture supernatant of dental pulp stem cells is substantially free from serum. For example, preferably, the serum content in the culture supernatant of dental pulp stem cells is 1% by mass or less, more preferably 0.1% by mass or less, even more preferably 0.01% by mass or less.

—Dental Pulp Stem Cells—

The dental pulp stem cells for the culture supernatant are not specifically limited. Stem cells from exfoliated deciduous teeth as well as deciduous dental pulp stem cells and permanent tooth dental pulp stem cells; DPSC) available in any other methods are usable.

Dental pulp-derived somatic stem cells can produce vascular endothelial cell growth factor (VEGF), hepatocellular growth factor (HGF), insulin-like growth factor (IGF), platelet-derived growth factor (PDGF), transforming growth factor-β (TGF-β)-1 and -3, TGF-a, KGF, HBEGF, SPARC, and other growth factors, as well as various cytokines such as chemokine. The cells can also produce a large number of other physiologically-active substances such as exosome.

In the present invention, especially preferably, the dental pulp stem cells for use for the culture supernatant of dental pulp stem cells are dental pulp stem cells containing many proteins, and more preferably, deciduous dental pulp stem cells are used. Specifically, in the cell growth method of the present invention, a cell growth medium containing a culture supernatant of deciduous dental pulp stem cells (SGF) is preferably used.

The dental pulp stem cells for use in the present invention may be natural ones or may also be genetically-modified ones so far as they can attain the intended treatment.

In the case where the somatic cells incubated in serum-free culture using the cell growth agent of the present invention or according to the cell growth method of the present invention are used for regenerative medicine, at the request by the Act on Securement of Safety of Regenerative Medicine Products, the culture supernatant of dental pulp stem cells for use in the agent or the method does not contain any other somatic stem cells than dental pulp stem cells. However, in the case where the cells incubated in serum-free culture using the cell growth agent of the present invention are used in any other technical field than regenerative medicine, the culture supernatant of dental pulp stem cells for use in the agent may contain mesenchymal stem cells or any other somatic stem cells than dental pulp stem cells, but preferably does not contain them. Especially preferably, the culture supernatant of dental pulp stem cells does not contain neural stem cells.

Other mesenchymal stem cells than dental pulp stem cells include, though not limited thereto, bone marrow stem cells, umbilical stem cells or adipose stem cells.

Examples of the other somatic stem cells than mesenchymal stem cells include, though not limited thereto, stem cells derived from dermal systems, digestive systems, myeloid systems or neural systems. Examples of dermal somatic stem cells include epithelial stem cells and hair follicle stem cells. Examples of digestive system somatic stem cells include pancreatic (overall) stem cells and hepatocellular stem cells. Examples of bone marrow somatic stem cells except mesenchymal stem cells include hematopoietic stem cells. Examples of neural system somatic stem cells include neural stem cells, and retinal stem cells.

The culture supernatant of dental pulp stem cells may contain any other stem cells than somatic stem cells but preferably does not contain them. Other stem cells than somatic stem cells include embryotic stem cells (ES cells), induced pluripotent stem cells (iPS cells) and embryonic carcinoma cells (EC cells).

—Method for Preparing Culture Supernatant of Dental Pulp Stem Cells—

With no specific limitation, any conventional method can be used as a method for preparing a culture supernatant of dental pulp stem cells.

A culture supernatant of dental pulp stem cells is a culture liquid obtained by incubation of dental pulp stem cells, and does not contain the cells themselves. For example, by separating and removing the cell fraction after incubation of dental pulp stem cells, a culture supernatant usable in the present invention can be obtained. A culture supernatant processed through various treatments (for example, centrifugation, concentration, solvent substitution, dialysis, freezing, drying, freeze-drying, dilution, desalting and storage) can also be used.

The dental pulp stem cells for preparing a culture supernatant of dental pulp stem cells can be sorted according to an ordinary method, for example, based on the size and the morphology of the cells, or as adherent cells. Dental pulp stem cells can be sorted from dental pulp cells collected from exfoliated primary teeth of permanent teeth, as adherent cells or subcultured cells thereof. As the culture supernatant of dental pulp stem cells, a culture supernatant obtained by incubating sorted stem cells can be used.

The “culture supernatant of dental pulp stem cells” is preferably a culture liquid not containing the cells themselves obtained by incubating dental pulp stem cells. Preferably, one embodiment of the culture supernatant of dental pulp stem cells for use in the present invention does not contain any cells (irrespective of kind of cells) as a whole. Owing to this characteristic feature, the cell growth agent of this embodiment is clearly differentiated not only from dental pulp stem cells themselves but also from various compositions containing dental pulp stem cells. One typical example of this embodiment is a composition composed of only a culture supernatant of dental pulp stem cells without containing dental pulp stem cells.

The culture supernatant of dental pulp stem cells for use in the present invention may contain a culture supernatant of both deciduous dental pulp stem cells and adult dental pulp stem cells. Preferably, the culture supernatant of dental pulp stem cells for use in the present invention contains a culture supernatant of deciduous dental pulp stem cells as an active ingredient, more preferably in an amount of 50% by mass or more, even more preferably 90% by mass or more. Especially preferably, the culture supernatant of dental pulp stem cells for use in the present invention is a composition composed of only a culture supernatant of dental pulp stem cells.

A basal medium or a basal medium added with serum or the like is usable as a culture liquid for dental pulp stem cells for obtaining a culture supernatant. The basal medium usable herein includes a Dulbecco's modified Eagle medium (DMEM), and in addition thereto, an Iscove's modified Dulbecco's medium (IMDM) (from GIBCO, etc.), a HamF12 medium (HamF12) (from SIGMA, GIBCO, etc.), and an RPMI1640 medium. Two or more kinds of basal media can be used as combined. Examples of mixed media include a medium prepared by mixing equal parts of IMDM and HamF12 (for example, commercially sold as a trade name: IMDM/HamF12 (from GIBCO)). Examples of components that may be added to the medium include serum (bovine fetal serum, human serum, ovine serum), serum substitutes (e.g., Knockout Serum Replacement (KSR)), bovine serum albumin (BSA), antibiotics, various vitamins and various minerals.

For producing a serum-free “culture supernatant of dental pulp stem cells”, it is recommended to use a serum-free medium throughout the total production process or in the last or several subculture steps from the last of the production process. For example, by incubating dental pulp stem cells in a medium not containing serum (serum-free medium), a serum-free culture supernatant of dental pulp stem cells can be prepared. In a production process of one or several subculture steps, when the last or several subculture steps from the last are carried out in a serum-free medium, a serum-free culture supernatant of dental pulp stem cells can be prepared. On the other hand, by removing serum from the collected culture supernatant through dialysis or solvent substitution using columns, a serum-free culture supernatant of dental pulp stem cells can be prepared.

For incubation of dental pulp stem cells for preparing a culture supernatant, ordinary conditions can be employed directly as they are. The preparation method for a culture supernatant of dental pulp stem cells may be the same as those in the cell incubation method to be mentioned hereinunder, except that the steps of isolation and selection of stem cells are appropriately controlled depending on the kind of the stem cells. Isolation and selection of dental pulp stem cells depending on the kind of dental pulp stem cells can be appropriately carried out by anyone skilled in the art.

—Other Components Contained in Culture Supernatant of Dental Pulp Stem Cells—

The culture supernatant of dental pulp stem cells for use in the present invention may contain any other components than the culture supernatant of dental pulp stem cells, but preferably does not substantially contain any other components.

(Content of Culture Supernatant of Dental Pulp Stem Cells)

The cell growth medium can contain the culture supernatant of dental pulp stem cells (or the cell growth agent of the present invention) in an amount of, for example, 0.1 to 100% by mass relative to the total of the cell growth medium, but preferably in an amount of 5% by mass or more. More preferably the cell growth medium contains the culture supernatant of dental pulp stem cells (or the cell growth agent of the present invention) in an amount of 5 to 20% by mass relative to the total of the cell growth medium, even more preferably 7 to 15% by mass. Within the range, a higher cell growth promoting effect can be attained. In the case where a freeze-dried culture supernatant of dental pulp stem cells or a freeze-dried cell growth agent of the present invention is used, the amount of the culture supernatant of dental pulp stem cells or the cell growth agent of the present invention to be contained in the cell growth medium can be reduced relative to the total amount of the cell growth medium.

(Basal Medium)

Though not specifically limited, the basal medium for use in the cell growth medium generally contains components for growing cells, such as amino acids, vitamins and inorganic salts. Examples of the basal medium include an Eagle minimum essential medium (MEM), an alpha-Eagle minimum essential medium (a-MEM), a Dulbecco's modified Eagle medium (DMEM), and a HamF12 medium (HamF12).

The “basal medium” in this description indicates a cell growth medium before a culture supernatant of dental pulp stem cells is added thereto, and corresponds to the above-mentioned commercially-available basal media.

(Other Components)

The cell growth medium may contain any other components in addition to the culture supernatant of dental pulp stem cells (especially, the cell growth agent of the present invention) and a basal medium, in accordance with the kind and the object of the mesenchymal stem cells and the somatic cells to be incubated, within a range not detracting from the advantageous effects of the present invention. The other components include nutrient components, antibiotics and cytokines.

Examples of the nutrient components include fatty acids, and vitamins.

Examples of the antibiotics include penicillin, streptomycin, and gentamycin.

The cytokines include those described in JP 2018-023343A [0014] to [0020].

On the other hand, preferably, the cell growth medium for use in the present invention does not substantially contain serum (bovine fetal serum, human serum, ovine serum). Also preferably, the cell growth medium for use in the present invention does not substantially contain any conventional serum substitutes such as Knockout Serum Replacement (KSR). For example, the content of serum or a conventional serum substitute in the cell growth medium for use in the present invention is preferably 1% by mass or less, more preferably 0.1% by mass or less, even more preferably 0.01% by mass or less.

(Preparation Method for Cell Growth Medium)

A preparation method for the cell growth medium of the present invention is not specifically limited. A culture supernatant of dental pulp stem cells (especially the cell growth agent of the present invention) may be produced according to the above-mentioned production method, and subsequently the resultant culture supernatant of dental pulp stem cells may be added to a commercially-available basal medium to prepare a cell growth medium. Alternatively, a commercially-available culture supernatant of dental pulp stem cells (especially the cell growth agent of the present invention) may be added to a commercially-available basal medium to prepare a cell growth medium. Further, a composition containing a culture supernatant of dental pulp stem cells that is to be discarded may be received (or such a composition is appropriately purified), and added to a commercially-available basal medium to prepare a cell growth medium.

The cell growth agent alone of the present invention may be used as a cell growth medium, directly as it is.

<Somatic Cells>

The animal species for the somatic cells for serum-free culture according to the cell growth method of the present invention is not specifically limited, and for example, humans, rats, mice and swine can be used depending on the intended use of the somatic cells. Preferably, the animal species for the somatic cells for serum-fee incubation is human.

The kind of the somatic cells for serum-free culture according to the cell growth method of the present invention is not specifically limited, and any arbitrary somatic cells can be serum-free incubated. Examples of the somatic cells for serum-free culture include ectomorphic cells, mesomorphic cells, endomorphic cells, and cells contained in a step of differentiating into these cells from fertilized eggs.

Examples of the ectomorphic cells include neuron cells, astrocyte cells, oligodendrocyte cells, and epidermal cells. Examples of the epidermal cells include epidermal keratinocytes.

Examples of the mesomorphic cells include vascular cells, hematopoietic lineage cells, mesenchymal cells, and dermal cells.

Examples of the hematopoietic lineage cells include hematopoietic precursor cells, erythrocytic cells, lymphocytic cells, granulocytic cells, and platelet cells.

Examples of the mesenchymal cells include bone cells, chondrocytic cells, muscular cells, myocardial cells, tendon cells, adipose cells, dermal papilla cells, pulpal cells, and fibroblasts.

Examples of the endomorphic cells include hepatic cells, pancreatic exocrine cells, pancreatic endocrine cells, and gallbladder cells.

In the case of incubating somatic cells for use in regenerative medicine or cell therapy according to the cell growth method of the present invention, somatic cells such as fibroblasts, epidermal keratinocytes, umbilical cells and adipose cells are incubated.

From the viewpoint of the advantageous effects of the present invention and from the viewpoint of collecting more cells, the somatic cells for serum-free culture are more preferably fibroblasts, epidermal keratinocytes, umbilical cells or adipose cells, and fibroblasts or epidermal keratinocytes are especially preferred.

A preparation method for somatic cells is not specifically limited, and can be appropriately selected in accordance with the intended object. For example, somatic cells can be prepared according to a method of isolating them from individuals such as human and mice, or a method of getting already-cloned cells from various organizations.

In the case where somatic cells are incubated according to the cell growth method of the present invention and used in therapy of returning them into bodies of patients suffering from various diseases, preferably, the somatic cells collected from the body of the patient are used.

<Cell Incubation Method>

In the cell growth method of the present invention, somatic cells are sowed in a cell growth medium and incubated therein by serum-free culture.

The cell incubation method of growing and incubating somatic cells is described.

As incubation conditions for somatic cells, preferred conditions may be appropriately employed depending on the kind of the somatic cells.

In the case of incubating somatic cells, preferably, somatic cells are incubated in an incubator under the conditions of, for example, a temperature of 37° C. and 5% CO₂. Regarding the incubation time, preferably, the cells are subcultured before reaching a confluent state. The passage number in subculture of somatic cells is not specifically limited, and may be appropriately selected depending on the intended purpose.

The resultant cell culture liquid, or the cell fraction obtained by separating cells alone from the cell culture liquid can be used, for example, in various cell therapies depending on the kind of the cells.

In the case where somatic cells are grown and incubated according to the cell growth method of the present invention, a cell growth medium to be mentioned below may be first prepared and somatic cells may be grown and incubated in the resultant medium.

<Use>

The cell growth medium for use in the present invention or the cell growth agent of the present invention can be used as a serum substitute for serum-free culture of somatic cells. As compared with conventional serum or serum substitutes, the cell growth medium for use in the present invention or the cell growth agent of the present invention have various advantages that they can be readily mass-produced, culture liquids of stem cells that have heretofore been discarded as industrial wastes can be effectively utilized, and the disposal cost for culture liquids of stem cells can be reduced. In particular, in the case where the culture supernatant of dental pulp stem cells is a culture supernatant of human dental pulp mesenchymal stem cells and where the somatic cells grown using the cell growth method of the present invention are applied to humans, other advantages are that the somatic cells are highly safe from the viewpoint of immunological aspects and have ethically fewer problems. In the case where the culture supernatant of dental pulp stem cells is a culture supernatant of dental pulp stem cells from patients suffering from various diseases and where the somatic cells grown using the cell growth method of the present invention are applied to the patients, the safety is higher and ethical problems reduce more.

Preferably, in the cell growth method of the present invention, somatic cells are grown and incubated without being differentiated. The cell growth method of the present invention differs from a method of growing cells through differentiation such as a conventional neurosphere method, that is, in the cell growth method of the present invention, cells can be grown and incubated without being differentiated.

A culture supernatant of dental pulp stem cells such as SGF for use in the cell growth agent of the present invention or in the cell growth method of the present invention contains a culture supernatant of dental pulp stem cells and therefore can be used in repairing therapy. In particular, a liquid containing SGF is favorably used in repairing therapy. Here, it is known that, in regenerative medicine predicated on stem cell transplantation, stem cells are not a leading part in regeneration, and the fluid element produced by stem cells can repair body organs along with the autochtonous stem cells. The present invention can solve difficult problems of cancerization, standardization, administration, preservation and incubation accompanied by conventional stem cell transplantation, and enables repairing therapy with a culture supernatant of dental pulp stem cells such as SGF. As compared with stem cell transplantation, repairing using a culture supernatant of dental pulp stem cells such as SGF does not require cell transplantation and therefore hardly causes cancerization and can be said to be safer. Further, another advantage of the present invention is that SGF having a constantly standardized quality can be used. Mass-production and efficient administration can be selected, and therefore the present invention can be utilized for various diseases at low cost. An administration method for SGF is drip infusion, local administration or nasal administration and is minimally invasive, and is almost free of untoward effects. For a local administration method, electroporation is preferred, in which fine pores are temporally formed through a cell membrane by applying a voltage (electric pulse) to a skin surface, and an active ingredient is penetrated into the dermic layer which could not be reached in ordinary care. Diseases that can be expected to have a reduced risk of developing disease symptoms owing to the effect of SGF administration include diabetes, liver ailment, kidney trouble, atopy, rheumatism (joint pain), ED (erectile dysfunction), vascular endothelial damage owing to hypertension, knee osteoarthritis, and infarction disorders such as cerebral infarction after-trouble. Further, antiaging is expected, such as amelioration and prevention of wrinkles and sagging/wound healing/whitening/hair growth and restoration/antioxidation. The administered SGF circulates in a body, and when a damaged tissue is found out, stem cells themselves are activated to repair and regenerate the tissue owing to the homing effect thereof. Further, the pituitary gland is stimulated to restore the hormone balance, and the metabolism cycle is thereby restored to the original metabolic state.

### EXAMPLES

Hereinunder the characteristic features of the present invention are described more specifically with reference to Examples and Comparative Examples given below. In the following Examples, the material used, its amount and ratio, the details of the treatment and the treatment process may be suitably modified or changed not overstepping the spirit and the scope of the invention. Accordingly, the invention should not be limitatively interpreted by the Examples mentioned below.

### Example 1

### <Preparation of Culture Supernatant of Dental Pulp Stem Cells>

According to the method described in Example 6 in JP 6296622, in which, however, a DMEM medium was used in place of the DMEM/HamF12 mixed medium, a culture supernatant of dental pulp stem cells was prepared. In primary culture, fetal bovine serum (FBS) was added to the medium to incubate, and in subculture, a supernatant of the subculture liquid that had been incubated using the primary culture liquid was separated so as not contain FBS therein, and used for preparation of a culture supernatant of deciduous dental pulp stem cells. DMEM is a Dulbecco's modified Eagle medium, and F12 is a HamF12 medium.

The resultant culture supernatant of deciduous dental pulp stem cells is SGF.

<Incubation of Fibroblasts Using Culture Supernatant>

A 10 mass % SGF medium was prepared, containing a DMEM medium and a cell growth agent of SGF in an amount of 10% by mass relative to the entire medium.

As somatic cells to be grown and incubated, human normal fibroblasts (HDF) were used. Human normal fibroblasts were sowed in the 10 mass % SGF medium in a 6-well plate in a ratio of 1×10⁵ cells/well. Plural samples were sowed so as to measure the absorbance after 3 days, 5 days and 7 days. The culture liquid was controlled to be 3 ml/well.

Under humidification and under pressure at 37° C. and 5% CO₂, the human normal fibroblasts were incubated.

<Quantitative Assay of Cell Growth (Cell Proliferation Potency Test)>

Using Premix WST-1 Cell Proliferation Assay System (from Takara Bio Inc.), cell growth quantitative assay was carried out through colorimetry according to the following process.

After the term of cell incubation, Premix WST-1 was added to each well in an amount of 300 μl/well, and further incubated under humidification and under pressure at 37° C. and 5% CO₂ for 1 hour to give a reaction liquid.

100 μl of the reaction liquid was transferred to a 96-well plate, and using a microplate reader (trade name Multiskan FC, from Thermoscientific Corporation), the absorbance at a wavelength of 450 nm of the background control and the sample was measured.

### [Comparative Example 1]: Serum-Free

As a control group, human dental pulp stem cells were incubated and assayed (12 samples in each test) in the same manner as in Example 1 except that a DMEM medium (serum-free) was used in place of the 10 mass % SGF medium.

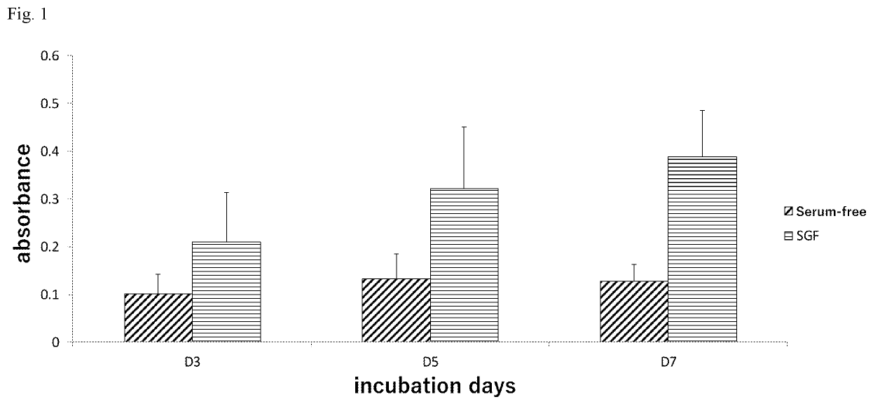

The results of cell growth quantitative assay in Example 1 (SGF) and Comparative Example 1 (serum-free) are shown in FIG. 1. In the graph, D3, D5 and D7 each indicate absorbance data of the reaction liquid in colorimetry of the culture liquid after the incubation days (cell incubation term) of 3 days, 5 days and 7 days, respectively, from the start of incubation.

As in FIG. 1, a significant difference is seen on a significance level of 1% or less between serum-free and SGF, and SGF exhibited a higher somatic cell growth promoting effect.

### [Comparative Example 2]: Culture Supernatant of Adipose Stem Cells (AT)

A culture supernatant of adipose stem cells was prepared according to Example 1, in which, however, human adipose stem cells were used in place of deciduous dental pulp stem cells. A 10 mass % AT medium was prepared, containing a DMEM medium and the culture supernatant of adipose stem cells in an amount of 10% by mass relative to the entire medium.

In the same manner as in Example 1 except that the 10 mass % AT medium was used in place of the 10 mass % SGF medium, human normal fibroblasts were incubated and assayed (12 samples in each test).

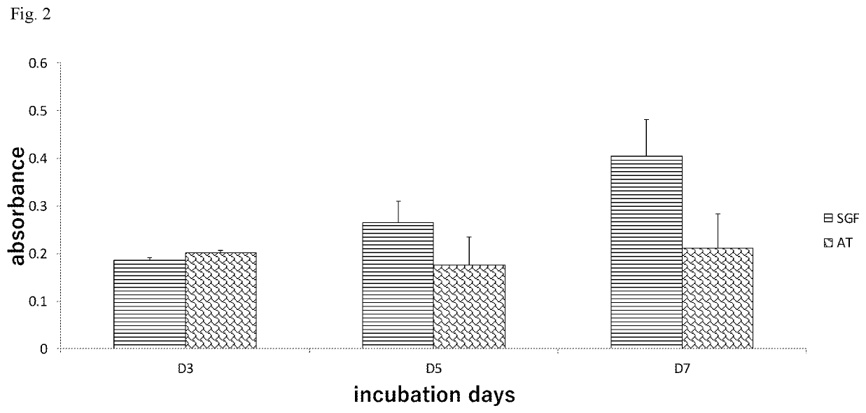

The results of cell growth quantitative assay in Example 1 (SGF) and Comparative Example 2 (AT) are shown in FIG. 2. As in FIG. 2, a significant difference is seen on a significance level of 1% or less between AT and SGF, and SGF exhibited a higher somatic cell growth promoting effect.

### [Comparative Example 3]: Culture Supernatant of Umbilical Stem Cells (UC)

A culture supernatant of umbilical stem cells was prepared according to Example 1, in which, however, human umbilical stem cells were used in place of deciduous dental pulp stem cells. A 10 mass % UC medium was prepared, containing a DMEM medium and the culture supernatant of umbilical stem cells in an amount of 10% by mass relative to the entire medium.

In the same manner as in Example 1 except that the 10 mass % UC medium was used in place of the 10 mass % SGF medium, human normal fibroblasts were incubated and assayed (9 samples in each test).

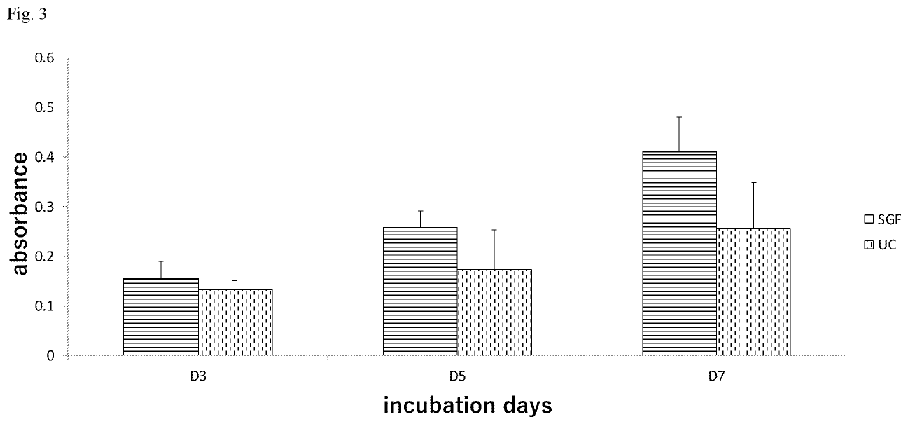

The results of cell growth quantitative assay in Example 1 (SGF) and Comparative Example 3 (UC) are shown in FIG. 3. As in FIG. 3, a significant difference is seen on a significance level of 1% or less between UC and SGF, and SGF exhibited a higher somatic cell growth promoting effect.

### [Comparative Example 4]: HFDM Medium

In the same manner as in Example 1 except that a commercially-available HFDM-1 medium (from Research Institute for the Functional Peptides Co., Ltd.) was used in place of the 10 mass % SGF medium, human normal fibroblasts were incubated and assayed (6 samples in each test).

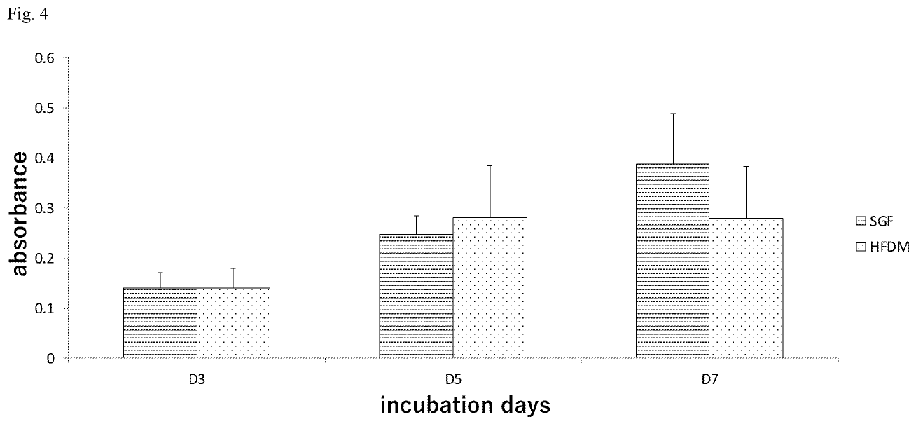

The results of cell growth quantitative assay in Example 1 (SGF) and Comparative Example 4 (HFDM) are shown in FIG. 4. As in FIG. 4, there was no significant difference between HFDM and SGF.

### [Comparative Example 5]: KBM Medium

In the same manner as in Example 1 except that a commercially-available KBM FibroAssist medium (from Kohjin Bio Co., Ltd.) was used in place of the 10 mass % SGF medium, human normal fibroblasts were incubated and assayed (6 samples in each test).

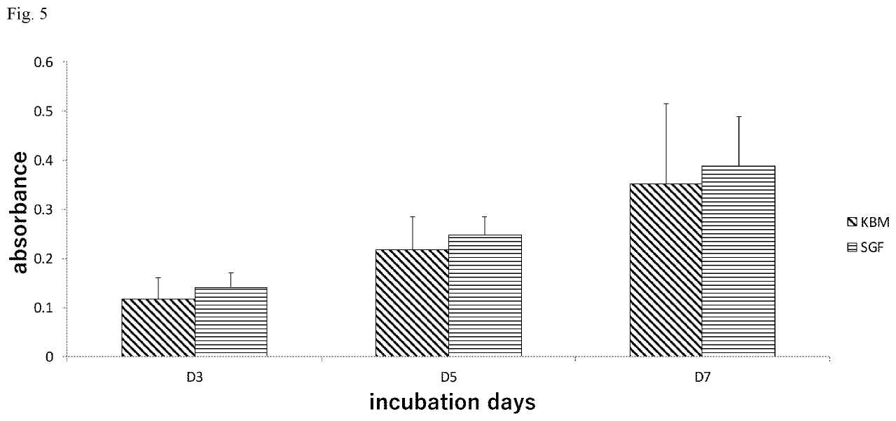

The results of cell growth quantitative assay in Example 1 (SGF) and Comparative Example 5 (KBM) are shown in FIG. 5. As in FIG. 5, there was no significant difference between KBM and SGF.

### [Comparative Example 6]: Fetal Bovine Serum (FBS)-Containing Medium

A 10 mass % FBS medium was prepared, containing a DMEM medium and fetal bovine serum (FBS) in an amount of 10% by mass relative to the entire medium.

In the same manner as in Example 1 except that the 10 mass % FBS medium was used in place of the 10 mass % SGF medium, human normal fibroblasts were incubated and assayed (12 samples in each test).

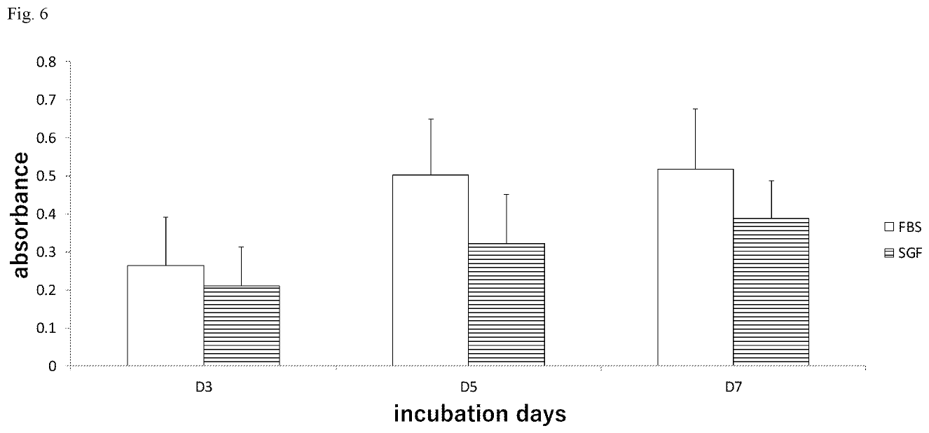

The results of cell growth quantitative assay in Example 1 (SGF) and Comparative Example 6 (FBS) are shown in FIG. 6. As in FIG. 6, there was no significant difference between FBS and SGF.

[Summary of Incubation Results of Fibroblasts]

As in FIG. 1 to FIG. 3, according to the present invention, the medium added with a culture supernatant of human dental pulp stem cells (SGF) exhibited a significant somatic cell growth promoting effect, as compared with the serum-free control medium of Comparative Example 1, the medium added with a culture supernatant of adipose stem cells of Comparative Example 2 (AT), and the medium added with a culture supernatant of umbilical stem cells of Comparative Example 3 (UC).

As in FIG. 4 to FIG. 6, according to the present invention, the medium added with a culture supernatant of human dental pulp stem cells (SGF) exhibited a somatic cell growth promoting effect comparable to the commercially-available medium (HFDM) of Comparative Example 4, the commercially-available medium (KBM) of Comparative Example 5, and the medium added with FBS of Comparative Example 6.

From these, it is known that the culture supernatant of human dental pulp stem cells is usable as a serum substitute for serum-free culture of somatic cells.

### [Example 101]: Incubation of Epidermal Keratinocytes Using Culture Supernatant of Dental Pulp Stem Cells

Human epidermal keratinocytes were incubated and assayed in the same manner as in Example 1 except that human epidermal keratinocytes were used as somatic cells to be grown and incubated.

### [Comparative Example 101]: Incubation of Epidermal Keratinocytes Using Serum-Free Medium

Human epidermal keratinocytes were incubated in a serum-free medium and assayed in the same manner as in Comparative Example 1, in which, however, human epidermal keratinocytes were used as somatic cells to be grown and incubated.

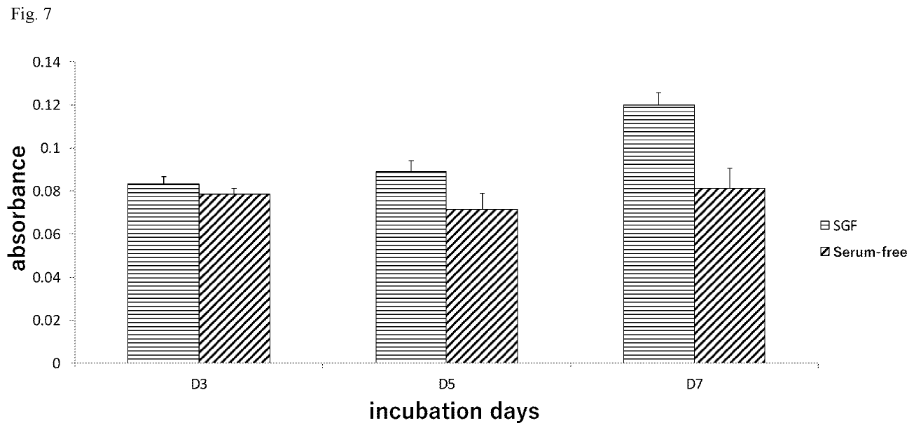

The results of cell growth quantitative assay in Example 101 (SGF) and Comparative Example 101 (serum-free) are shown in FIG. 7. As in FIG. 7, a significant difference is seen on a significance level of 1% or less between serum-free and SGF in incubation of epidermal keratinocytes.

### [Comparative Example 102]: Incubation of Epidermal Keratinocytes Using Culture Supernatant of Umbilical Stem Cells (UC)

Human epidermal keratinocytes were incubated in a medium containing a culture supernatant of umbilical stem cells (UC) and assayed in the same manner as in Comparative Example 3, except that human epidermal keratinocytes were used as somatic cells to be grown and incubated.

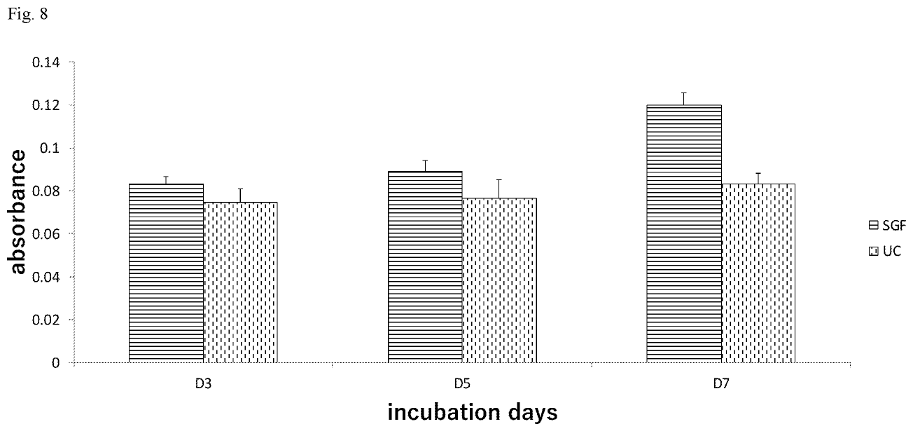

The results of cell growth quantitative assay in Example 101 (SGF) and Comparative Example 102 (UC) are shown in FIG. 8. As in FIG. 8, a significant difference is seen on a significance level of 1% or less between UC and SGF in incubation of epidermal keratinocytes.

### [Comparative Example 103]: Incubation of Epidermal Keratinocytes Using Commercially-Available Serum-Free Medium

Human epidermal keratinocytes were incubated and assayed in the same manner as in Example 101, except that a commercially-available serum-free medium, Keratinocyte Growth Medium 2 Kit (from Takara Bio Inc.) was used in place of the 10 mass % SGF medium.

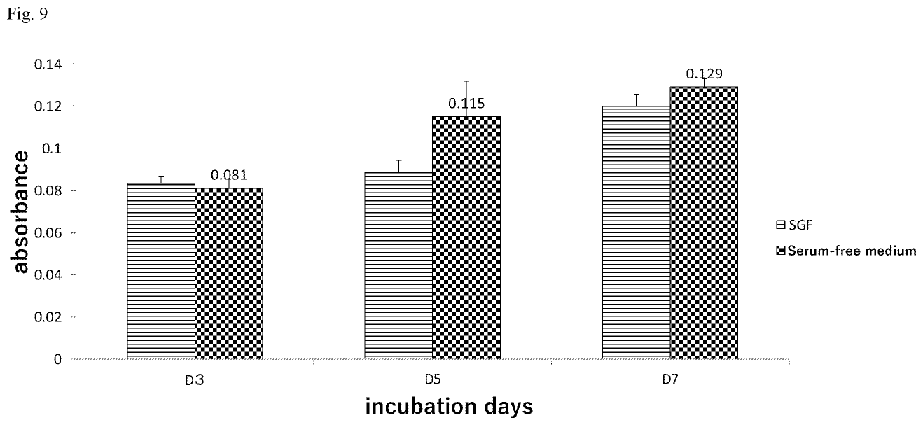

The results of cell growth quantitative assay in Example 101 (SGF) and Comparative Example 103 (serum-free medium) are shown in FIG. 9. As in FIG. 9, there was seen no significant difference between the commercially-available serum-free medium and SGF.

### [Comparative Example 104]: Incubation of Epidermal Keratinocytes Using Fetal Bovine Serum (FBS)-Containing Medium

Using a 10 mass % FBS medium, human epidermal keratinocytes were incubated and assayed in the same manner as in Comparative Example 6, except that human epidermal keratinocytes were used as somatic cells to be grown and incubated.

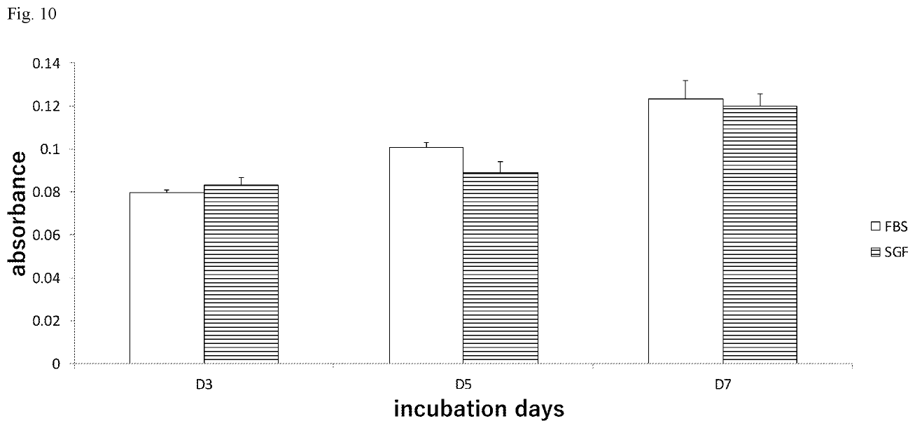

The results of cell growth quantitative assay in Example 101 (SGF) and Comparative Example 104 (FBS) are shown in FIG. 10. As in FIG. 10, there was seen no significant difference between FBS and SGF in incubation of epidermal keratinocytes.

[Summary of Incubation Results of Epidermal Keratinocytes]

As in FIG. 7 and FIG. 8, according to the present invention, the medium added with a culture supernatant of human dental pulp stem cells (SGF) exhibited a significant somatic cell growth promoting effect, as compared with the serum-free control medium of Comparative Example 101, and the medium added with a culture supernatant of umbilical stem cells of Comparative Example 102 (UC).

As in FIG. 9 and FIG. 10, according to the present invention, the medium added with a culture supernatant of human dental pulp stem cells (SGF) exhibited a somatic cell growth promoting effect comparable to the commercially-available medium (serum-free medium) of Comparative Example 103, and the medium added with FBS of Comparative Example 104.

From these, it is known that the culture supernatant of human dental pulp stem cells is usable as a serum substitute for serum-free culture of somatic cells.

### Example 201

### <Growth and Incubation of Deciduous Dental Pulp Stem Cells Using Culture Supernatant of Deciduous Dental Pulp Stem Cells>

A 10 mass % SGF medium was prepared, containing a DMEM medium and a cell growth agent of SGF in an amount of 10% by mass relative to the entire medium.

As somatic cells to be grown and incubated, the same fibroblasts as in Example 1 were used, and the fibroblasts were incubated and assayed (3 samples in each test).

### Comparative Example 201

### <Growth and Incubation of Deciduous Dental Pulp Stem Cells Using Culture Supernatant of Permanent Dental Pulp Stem Cells>

In the same manner as in Examples in CN 105861429, adult exfoliated and extracted healthy teeth were collected to prepare adult permanent dental pulp stem cells.

According to Example 1 except that adult permanent dental pulp stem cells were used in place of the deciduous dental pulp stem cells, a culture supernatant of adult permanent dental pulp stem cells was prepared. A 10 mass % adult permanent dental pulp stem cell medium was prepared, containing a DMEM medium and a culture supernatant of adult dental pulp stem cells in an amount of 10% by mass relative to the entire medium.

In the same manner as in Example 201 except that the 10 mass % adult dental pulp medium was used in place of the 10 mass % SGF medium, fibroblasts were incubated and assayed (3 samples in each test).

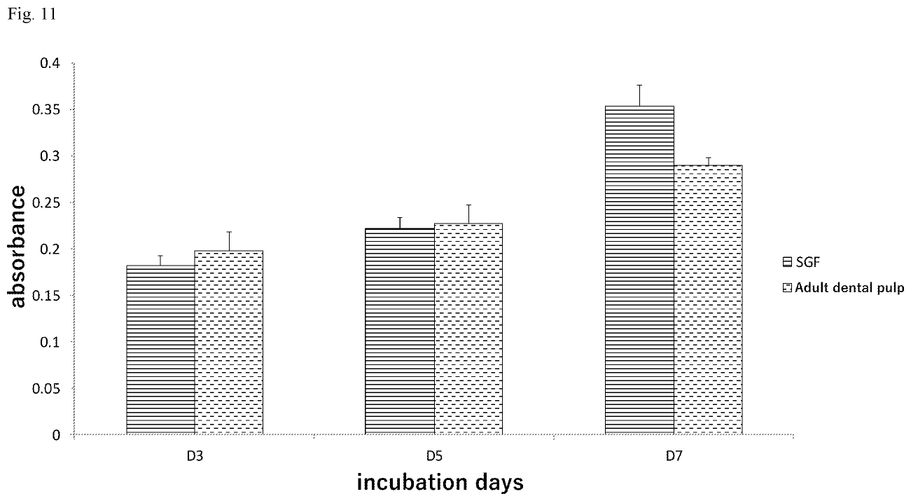

FIG. 11 shows the results of quantitative assay of cell growth in Example 201 (SGF) and Comparative Example 201 (adult dental pulp). As in FIG. 11, the somatic cell growth promoting effect was more remarkable on Day 7 of incubation using the deciduous dental pulp stem cells-derived SGF than using the adult dental pulp stem cells.

From this, it is known that the culture supernatant of deciduous dental pulp stem cells exhibits a more remarkable somatic cell growth promoting effect in serum-free incubation of somatic cells than the culture supernatant of adult permanent dental pulp stem cells.

The present disclosure relates to the subject matter contained in Japanese Patent Application No. 2019-100248 filed on May 29, 2019 and Japanese Patent Application No. 2019-220646 filed on Dec. 5, 2019, the contents of which are expressly incorporated herein by reference in their entirety. All the publications referred to in the present specification are also expressly incorporated herein by reference in their entirety.

The foregoing description of preferred embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined claims.

What is claimed is:

1. A cell growth method including serum-free culture of somatic cells sowed in a cell growth medium containing a culture supernatant of dental pulp stem cells, wherein
the somatic cells exclude physically or physiologically defected somatic cells.

2. The cell growth method according to claim 1, wherein only a cell growth agent containing a culture supernatant of deciduous dental pulp stem cells is used as the culture supernatant of dental pulp stem cells.

3. The cell growth method according to claim 1, wherein the somatic cells are fibroblasts or epidermal keratinocytes.

4. A cell growth agent containing a culture supernatant of deciduous dental pulp stem cells,
for use for serum-free culture of somatic cells, wherein
the somatic cells exclude physically or physiologically defected somatic cells.

5. A cell growth medium containing the cell growth agent according to claim 4,
for use for serum-free culture of somatic cells, wherein
the somatic cells exclude physically or physiologically defected somatic cells.

6. The cell growth medium according to claim 5, containing the cell growth agent in an amount of 5% by mass or more relative to the total of the cell growth medium.
